(19) United States
(12) Patent Application Publication
(10) Pub. No.: US 2014/0135657 A1
(43) Pub. Date: May 15, 2014

(54) Management system and method for damage risk of tissue pressure

(71) Applicant: INDUSTRIAL TECHNOLOGY RESEARCH INSTITUTE, HsinChu (TW)

(72) Inventors: Chi-Kang Wu, Taipei City (TW); Tsai-Ya Lai, Hsinchu City (TW); Hsiao-Wei Yeh, Hsinchu County (TW); Yu-Ting Tseng, Hsinchu City (TW)

(73) Assignee: INDUSTRIAL TECHNOLOGY RESEARCH INSTITUTE, HsinChu (TW)

(21) Appl. No.: 13/965,410

(22) Filed: Aug. 13, 2013

(30) Foreign Application Priority Data
Nov. 13, 2012 (TW) 101142225

(51) Int. Cl. A61B 5/103

(52) U.S. Cl. CPC A61B 5/1038
USPC 600/592

(57) ABSTRACT

According to one embodiment of a management system for damage risk of tissue pressure, at least one pressure sensor is deployed on at least one pressure-withstanding location on a body surface of a user, and detects a plurality of extremity pressure signals from the at least a pressure-withstanding location. An information processing device uses the plurality of extremity pressure signals to compute and store at least one risk assessment index, uses a plurality of features of the plurality of extremity pressure signals to compute at least one risk adjustment factor, and then uses the at least one risk adjustment factor to calibrate the at least one risk assessment index.

### CROSS-REFERENCE TO RELATED APPLICATION

The present application is based on, and claims priority from, Taiwan Patent Application No. 101142225, filed Nov. 13, 2012, the disclosure of which is hereby incorporated by reference herein in its entirety.

### TECHNICAL FIELD

The present disclosure relates generally to a management system and method for damage risk of tissue pressure.

### BACKGROUND

According to the statistics of the relevant information, the withstanding loading amount of foot for human activities is shocking. The peak value of the vertical force is about 120% of the body weight when walking and about 275% of body weight when running. In recent years, research of plantar pressure pointed out that the loading distribution for the plantar weight of individual bare foot when standing is about 60% at the heel, about 8% at the midfoot, about 28% at the forefoot, and about 4% at the toes. The pressure peak at the heel is about 2.6 times large than that at the forefoot. The pressure peak at the forefoot is generated at the bottom of the second metatarsal head. Therefore, in normal cases a normal foot for a normal person needs to withstand a considerable gravity, not to mention foot damage easily occurred in high-risk groups, such as diabetes populations, pregnant women, and smokers.

The design of customized insole is the mainstream technology for foot care of diabetes patients. Such technologies select different insole materials and designs based on disease conditions and foot characteristics. However, when the human body walks for a long time, the tissue state will change over time. The customized insole designed at the stationary state of the patient is unable to meet the foot characteristics of walking for a long time. If one can aware of abnormal changing of foot through continuing detection, then there may be opportunity of early prevention to greatly improve the quality of life of the patient, and therefore, most patients may also avoid facing amputation pain.

There are existing related technologies of pressure-withstanding sensing and estimating risk. For example, a technology uses a medical sensor to respond a predetermined condition. This predetermined condition such as is the obtained pressure-withstanding of the human body at an attached substrate. This medical sensor uses wireless communication to respond the induction of pressure-withstanding of the human body in order to avoid generation of pressure ulcer.

One technique provides a method for analyzing a gait pattern. As shown in

FIG. 1, this method firstly measures, by a plurality of force sensing resistor (FSR) sensors, foot pressure values (step 110), outputs the measured foot pressure values, respectively, (step 120); searches for a maximum pressure value outputted from the FSR sensors of a plurality of pressure local areas (step 130), and calculates a center of pressure (COP) with respect to the detected maximum pressure value of each local area (step 140), then analyzes a gait pattern by adding the calculated COP to the trajectory of COPs (step 150).

A technique provides a monitoring device for the risk modeling for pressure ulcer formation of the user. Its method of surface pressure accumulated exposure value firstly measures the integral value of the pressure versus time, and then uses a pressure sensor to derive pressure exposure or risk values, and normalizes these values through referring to user information, and displays the normalized pressure exposure or risk values in a graphical manner. Its assessing method for pressure ulcer formation firstly assesses a time threshold of reaching a high-risk under a certain pressure, and then adjusts the threshold according to composite physiological parameters. The composite physiological parameters include such as temperature, humidity, etc.

When the body is exposed to withstand external pressure, the circulatory system is oppressed and the local blood flow is reduced to lead to tissue necrosis. According to an exemplary relationship diagram in FIG. 2 of the skin pressure applied versus time for pressure ulcer formation of the human body, wherein the horizontal axis represents time (unit: hour), and the vertical axis represents the skin pressure applied (units: mm Hg), there is a hyperbolic relationship between the strength and the duration of the skin pressure applied, wherein the strength of skin pressure applied depends on both the soft tissue and the circulation state of the user.

In the above mentioned and existed related technologies for pressure sensing and assessing risk of a body surface, the technologies choosing different insole materials and designs according to the groups or constitution characteristics of patients or the users fail to monitor and respond to the long-term change of soft tissue characteristics of user foot, thus its design and structure may only be applied to particular patients within a specific time period, and unable to record and adapt to the patients' tissue change to adjust the care strategy to the patients. It may be seen that from the relationship diagram shown in FIG. 2, when a user occurs poor circulation condition or foot lesions, and this leads to the concentration of abnormal pressures, his/her envelope 210 of time vs. pressure-withstanding will go down. Therefore, there is a need to develop products that may continuously detect extremity pressure change and perform risk monitoring of tissue damage.

Therefore, it is needed to design a management technique for damage risk of tissue pressure, to assess the degree of the user's extremity exposure to tissue damage risk through a pressure sensing approach; and during performing risk analyzing of pressure change, the technique may put the related adjustment factors, such as age, different user groups into consideration; identify long-term pressure change of the user's extremity, such as orientation change of the plantar COP, whether occurring foot change of the high-risk area, and accordingly adjust risk parameters to assist patients self-aware of early symptoms and provide as clinical diagnosis reference to the physician.

### SUMMARY

The exemplary embodiments of the disclosure may provide a management system and method for damage risk of tissue pressure.

One exemplary embodiment relates to a management system for damage risk of tissue pressure. The system may comprise at least one pressure sensor and an information processing device. The at least one pressure sensor is deployed on at least one pressure-withstanding location on a body surface of a user, and detects a plurality of extremity pressure signals from the at least one pressure-withstanding location. The information processing device computes and temporarily stores at least one risk assessment index according to the plurality of extremity pressure signals, computes at least one risk adjustment factor according to a plurality of features of the plurality of extremity pressure signals, and then calibrates the at least one risk assessment index according to the at least one risk adjustment factor.

Another exemplary embodiment relates to a management method for damage risk of tissue pressure. The method may comprise: deploying at least one pressure sensor on at least one pressure-withstanding location on a body surface of a user, and detecting a plurality of extremity pressure signals from the at least one pressure-withstanding location; and using an information processing device to compute and storing at least one risk assessment index according to the plurality of extremity pressure signals, and compute at least one risk adjustment factor by using a plurality of features of the plurality of extremity pressure signals, and then calibrate the at least one risk assessment index according to the at least one risk adjustment factor.

### BRIEF DESCRIPTION OF THE DRAWINGS

FIG. 1 shows a flowchart for a method of analyzing gait pattern.

FIG. 2 shows a relationship diagram of skin pressure applied versus time for pressure ulcer formation of the human body.

FIG. 3 shows a schematic view for gait characteristics of the human body, according to an exemplary embodiment.

FIG. 4 shows a flow for recording plantar pressure risk factors and adjusting the risk factors, according to an exemplary embodiment.

FIG. 5 shows a management system for damage risk of tissue pressure, according to an exemplary embodiment.

FIG. 6 shows a schematic view of deploying a plurality of plantar pressure elements, according to an exemplary embodiment.

FIG. 7 shows a schematic view illustrating an up-adjustment of the human pressure ulcer risk, according to an exemplary embodiment.

FIG. 8A shows a schematic view illustrating the accumulated values of plantar pressure signals ΣP and the risk assessment index of the plantar pressures in FIG. 6, according to an exemplary embodiment.

FIG. 8B shows a schematic view illustrating the risk adjustment factors of plantar pressures in FIG. 6, according to an exemplary embodiment.

FIG. 9 shows a management method for damage risk of tissue pressure, according to an exemplary embodiment.

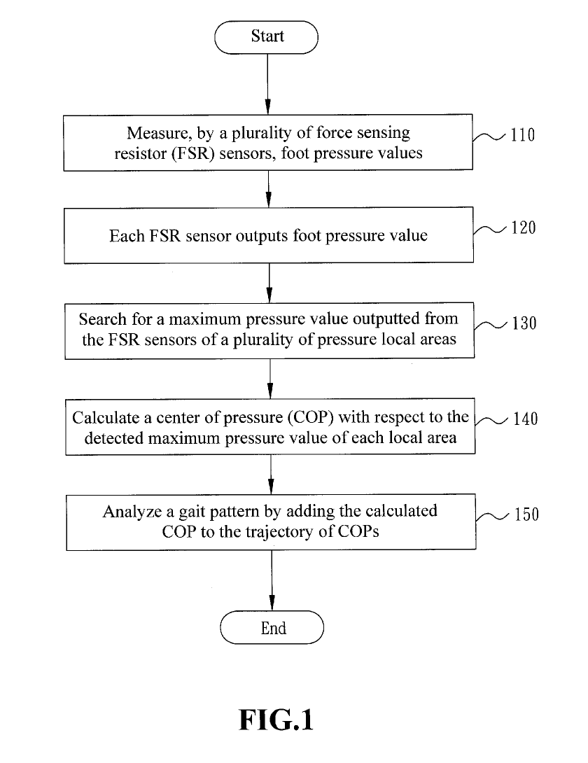

### DETAILED DESCRIPTION OF DISCLOSED EMBODIMENTS

Below, exemplary embodiments will be described in detail with reference to accompanying drawings so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

The disclosed exemplary embodiments of a management technique for damage risk of tissue pressure assess and display a damage risk index of the extremity tissue under the body surface for the animal having hard and soft tissues, such as human beings, and calculate risk adjustment factors according to the features of pressure signals, then adjust the tissue damage risk index according to the risk adjustment factors; wherein the body surface means the skin surface, soft tissue is, for example, extremity (foot, hand, and so on) of a human body. The following exemplary embodiments take the foot of the human body as an exemplar for illustration, but the scenario for the disclosed embodiments is not limited to this exemplar.

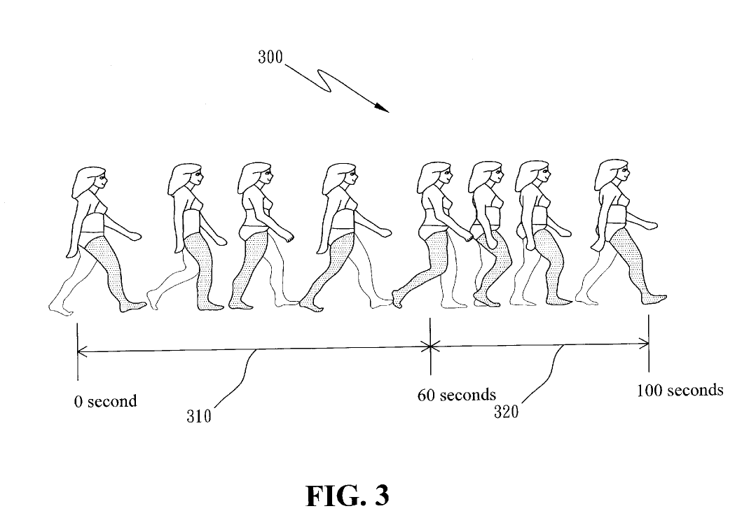

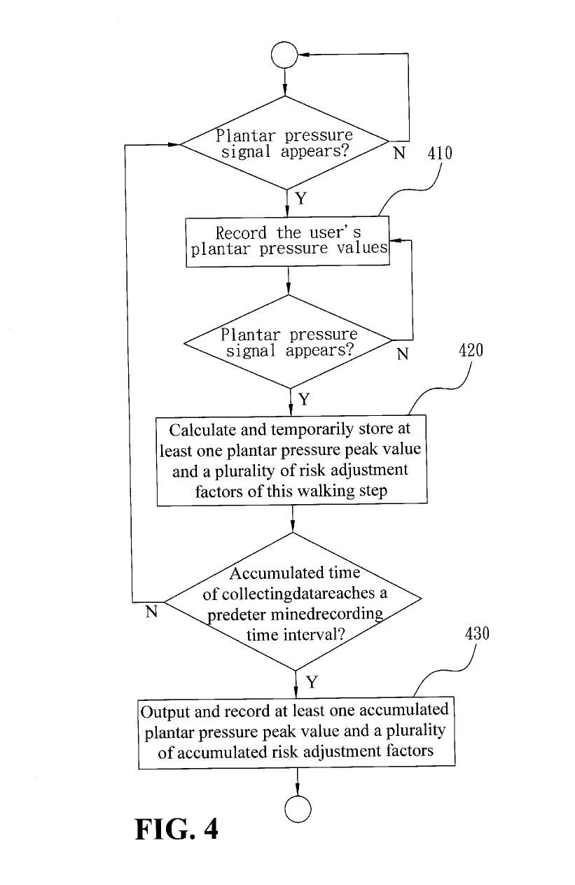

It may be seen from the gait characteristics diagram 300 of the human body in FIG. 3 that the left foot and the right foot must contain a stance phase 310 and a swing phase 320 in every step of the human natural walking. The standing phase 310, for example lasts from 0-60 seconds; and after 60 seconds, the vacant phase 320 occurs until 100 seconds. Since plantar tissue damage is caused by applying too much pressure on foot or withstanding too much time. Therefore, it is needed to analyze the pressure withstanding data of the plantar pressure in the stance phase. Thus, the period begins from the foot pressure sensor appearing signals (i.e. the plantar touches the ground to enter into the stance phase) to the signal of the extremity pressure sensor becoming zero (i.e., the planter leaves the ground to enter into the swing phase) is defined as a human walking step. Accordingly, FIG. 4 shows a flow for recording plantar pressure risk factors and adjusting the risk factors, according to an exemplary embodiment.

As shown in FIG. 4, during each walking step, when the extremity pressure sensor appears signal (this signal is called the plantar pressure signal) the exemplary embodiment of the present disclosure may begin recording the user's plantar pressure values (step 410), and when the sole leave off the ground (i.e., plantar pressure signal disappears), it may calculate and temporarily store at least one plantar pressure peak value and a plurality of risk adjustment factors of this walking step (step 420). Then, when the accumulated time of collecting data reaches a predetermined recording time interval, it may output and record at least one accumulated plantar pressure peak value and a plurality of accumulated risk adjustment factors (step 430). These risk adjustment factors may be derived by the features values of the plantar pressure signals through a machine learning system. The machine learning systems may be, but not limited to, such as the artificial neural network (artificial neural network) system, the support vector machine (SVM), etc.

In other words, at the beginning of the extremity (e.g., the foot, palm, etc.) pressure sensor appearing the extremity pressure signal, the exemplary embodiments of the present disclosure may record the extremity pressure signal values during each walking step, until there is no pressure signal appeared. An information processing device may be used to calculate and temporarily store an accumulated value of the extremity pressure and at least one risk adjustment factor of this walking step after the extremity leaves the ground. When the time of collecting the extremity pressure signals reaches a predetermined recording time interval, the stored plantar pressure peak values and the stored risk adjustment factors may be outputted, for example, to a communication device or a recording media.

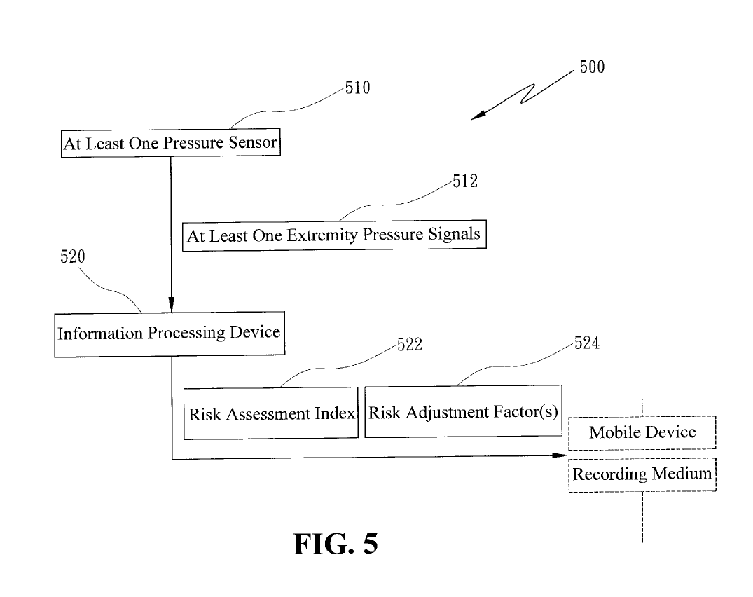

Accordingly, FIG. 5 shows a management system for damage risk of tissue pressure, according to an exemplary embodiment. Referring to FIG. 5, a management system for damage risk of tissue pressure 500 may comprise at least one pressure sensor 510, and an information processing device 520. The at least one pressure sensor 510 may be deployed on at least one pressure-withstanding location on a body surface of a user, such as at least one extremity location of the user, and detect a plurality of extremity pressure signals 512 from the at least one pressure-withstanding location on the body surface. The at least one pressure sensor 510 may be, but not limited to, the flexible electronic device. The information processing device 520 computes and temporarily stores at least one risk assessment index 522 according to the plurality of extremity pressure signals 512, and computes at least one risk adjustment factor 524 according to a plurality of features of the plurality of extremity pressure signals, and then calibrates at least one risk assessment index 522 according to the at least one risk adjustment factor 524. As the preceding described, when the time of collecting the extremity pressures reaches a predetermined time interval, the at least one risk assessment index 522 and the at least one risk adjustment factor 524 are outputted, for example, to a mobile device or a recording media. The user may be an animal (such as human being) having hard and soft tissues (such as extremities).

According to the exemplary embodiments, the information processing device 520 may use a wireless or a wired connection to connect a plurality of pressure sensors 510. The at least one risk assessment index 522 may be at least one extremity maximum pressure value of a user, and the at least one extremity maximum pressure value may be computed by adding a plurality of pressure peak values of one or more extremity pressure areas (e.g., the one or more plantar pressure areas of each walking step) of the user. The plurality of features of the plurality of extremity pressure signals 512 may be derived by a machine learning system such as the neural networks, the SVM, etc. The plurality of features may be, but not limited to, features of the extremity pressure center moving curve, the extremity pressure time change, the local pressure performance, or derived by the features of the left and the right extremity pressure performances of the extremities. The following detailed illustrates how to calculate or derive the at least one risk assessment index 522, the plurality of features of the extremity pressure signals 512, and the at least one risk adjustment factor 524. etc.

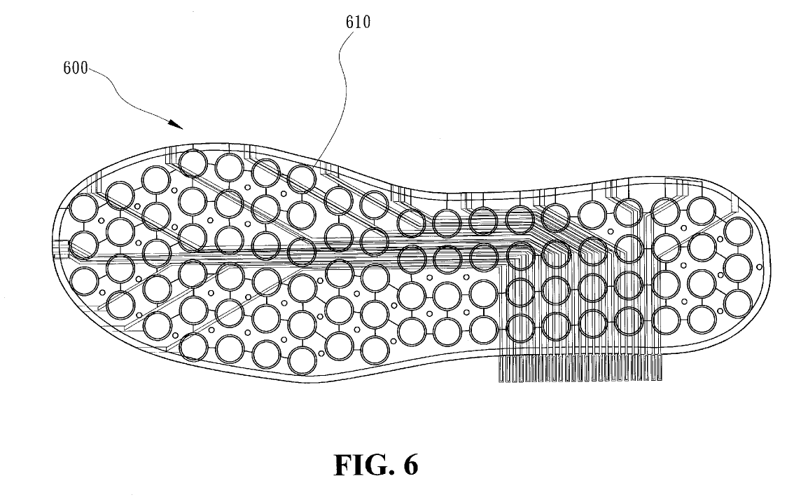

The followings take the foot as an exemplar of the extremity for illustration. When the management system for damage risk of tissue pressure 500 in the exemplary embodiments is applied, the plurality of pressure sensors 510 may be deployed on a pressure-withstanding area on a body surface of a user such as the user plantar. As shown in the exemplar of FIG. 6, a plurality of plantar pressure sensors are deployed on whole plantar 600, wherein each circle represents a deployed location of any plantar pressure sensor, as shown in label 610, and the areas deployed by the plurality of pressure sensor covers the whole plantar 600. The plantar pressure sensors, may be, but not limited to, a flexible electronic device that may be manufactured by flexible electronic technology, and attached to or embedded in an insole. Each plantar pressure sensor outputs the applied pressure size of an arbitrary point (not shown) on the insole. After an origin (not shown) is assigned, the distance d of the arbitrary point and the origin on the insole is found. The distance d may be de-composited into an X-direction component dx and a Y-direction component dy according to the definition of a coordinate system. Accordingly, pressure distribution points, for example P1 to P85 (not shown), on the insole, and the distance of each point to the insole dx1 to dx85 and dy1 to dy85 are obtained.

The information processing device 520 may distinguish every walking step of the foot steps, and continuously capture the user's plantar pressure information after the user begins the stance phase of every walking step, use plantar pressure signal to calculate the plantar pressure risk assessment index, the features of the plantar pressure signals, and the plantar pressure risk adjustment factor(s), etc.

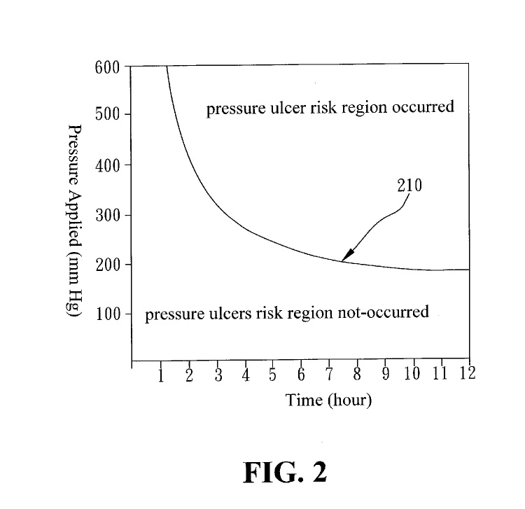

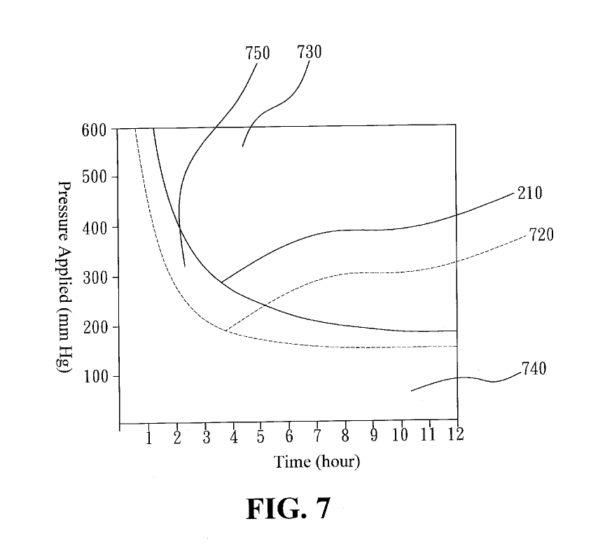

Then, the relationship diagram of the pressure-withstanding versus time shown in FIG. 2 may be used to assess whether the user is in the state of a pressure ulcer risk. When the user appears an abnormal circulatory state, such as the situation of edema or lower extremity vein blockage, the change of circulatory state may increase the pressure ulcers risk of the lower extremity. In other words, due to the poor circulatory state, or the foot lesions occurring change of the user causes a concentrated abnormal pressure, the envelope of time versus pressure-withstanding may further go downward. This phenomenon is as shown in FIG. 7, wherein the dashed line represents the envelope of the pressure ulcer risk 720 after downward. The upper area of the envelope 720 is the skin ulceration area 730, and the lower area of the envelope 720 is the no skin ulceration area 740. In other words, when the user appears an abnormal circulatory state, the pressure ulcer area may be enlarged, e.g., the risk of occurring pressure ulcer of the user may be increased. In the exemplar, due to the user appears an abnormal circulatory state, the enlarged pressure ulceration area is indicated by the label 750 as shown.

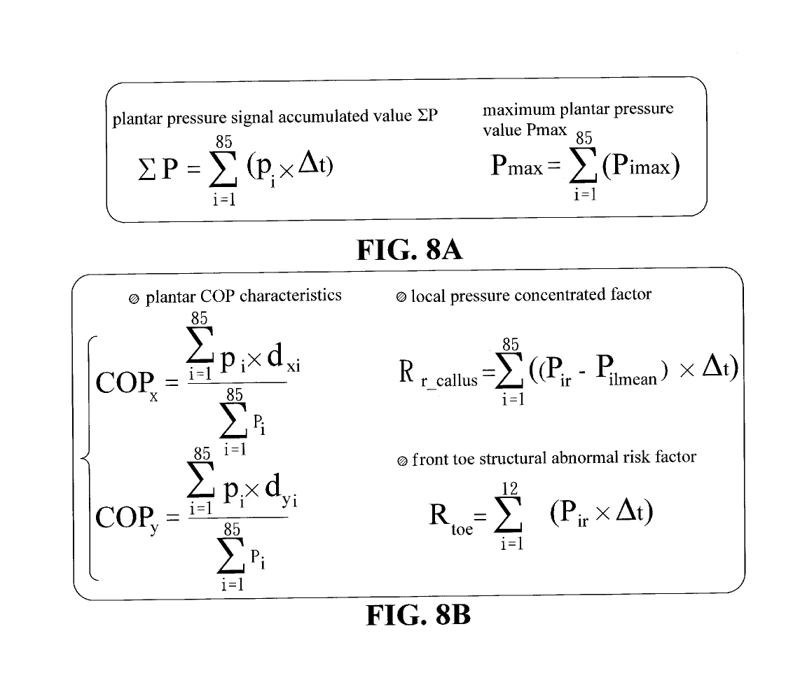

Take FIG. 6 as an exemplar, assuming that there are a total of 85 points of plantar pressure sources sensed by the plurality of plantar pressure sensors, then 85 values of plantar pressure signals P₁ to P₈₅ are obtained from these 85 points. An accumulated value ΣP of the plantar pressure signals is defined as follows:

$$\sum P = {\sum }_{i = 1}^{85} ( {p}_{i} \times Δ t )$$

Wherein, Δt represents the time difference of before and after capturing the pressure signals, the unit for example, is second, and pᵢ represents the obtained value of the plantar pressure signal Pᵢ from the plantar pressure sensor i. The ΣP may describe the accumulated value of the plantar pressure-withstanding versus time after adjusted to a time scale. The risk assessment index of the plantar pressure may be, but is not limited to, a maximum plantar pressure value Pmax of the user. The maximum plantar pressure value Pmax may replace the accumulated value ΣP of the plantar pressure signal. While the maximum plantar pressure value Pmax may be calculated by summing up the plurality of pressure peak values of a plurality of plantar pressure areas, that is, calculated by summing up each pressure peak value Pimax of each plantar pressure sensor i, i.e., the total sum of the pressure peak values of all plantar pressure sensors. In FIG. 6, for example, the maximum plantar pressure value Pmax is calculated by summing up each pressure peak value Pimax of each plantar pressure sensor i of 85 plantar pressure sensors. Accordingly, FIG. 8A shows a schematic view illustrating the accumulated values of plantar pressure signals ΣP and the risk assessment index of the plantar pressures in FIG. 6, according to an exemplary embodiment.

The aforementioned risk upper-adjusted degree caused by the abnormal circulatory state in FIG. 7 may be assessed by using a drift distance of calculating the center of pressure (COP) of the extremity pressure. To calculate the drift distance of the COP of the extremity pressure, one needs to firstly calculate the COP location of the extremity pressure. The displayed deployment of the plantar pressure sensors in FIG. 6 is taken as an exemplar for illustration. Since there are a total of 85 points of plantar pressure signal sources, thus there are a total of 85 plantar pressure signal values p₁ to p₈₅. The information from FIG. 6 may be used to calculate the X-direction component COPₓ and the Y-direction component COP_{y} of the COP location of the plantar according to the following formula:

$$C O {P}_{x} = \frac{{\sum }_{i = 1}^{85} {P}_{i} \times {d}_{xi}}{{\sum }_{i = 1}^{85} {p}_{i}}$$

$$C O {P}_{y} = \frac{{\sum }_{i = 1}^{85} {P}_{i} \times {d}_{yi}}{{\sum }_{i = 1}^{85} {p}_{i}}$$

Wherein pᵢ represents the obtained plantar pressure signal value from the plantar pressure sensor i, the dₓᵢ represents the X-direction component of the distance d between the location point of the plantar pressure sensor i and the origin point O, the d_{yi} represents the Y-direction component of the distance d between the location point of the plantar pressure sensor i and the origin point O.

After the COP location has been calculated, an extreme value of the COP for every walking step in the Y direction may be recorded, and the extreme value of the COP for every walking step in the Y direction for each step may be defined as the drift distance of the COP for the plantar pressure in the Y direction, and which may act as an adjustment factor for assessing the plantar pressure risk.

According to the exemplary embodiments, in addition to the X-direction component COPₓ and the Y-direction component COP_{y} of the COP location of the sole may act as the risk adjustment factor for the plantar, a local pressure concentrated factor R_callus and a toe structural abnormal risk factor Rtoe may also be taken into consideration. The local pressure concentrated factor R_callus describes the abnormal circulatory state and the soft tissue hyperplasia; and the toe structural abnormal risk factor Rₜₒₑ describes the toes structure abnormality and the toe joint abnormality. Take FIG. 6 as an exemplar, the right foot local pressure concentrated factor Rᵣ_{_{—}}_{callus} and the fore toe structural abnormal risk factor Rₜₒₑ may be calculated according to the following formula:

$${R}_{r\_callus} = {\sum }_{i = 1}^{85} ( ( {P}_{ir} - {P}_{ilmean} ) \times Δ t ) , \\ {R}_{toe} = {\sum }_{i = 1}^{12} ( {p}_{i} \times Δ t )$$

wherein P^{ir} is the pressure value of the right foot plantar pressure sensor i, Pᵢₗₘₑₐₙ is the average pressure value of the left foot of the plantar pressure sensor i in the previous walking step, Δt represents the time difference of before and after capturing pressure signal, and the unit, for example, is second. The left foot local pressure concentrated risk adjustment factor Rₗ_{_{—}}_{callus} replaces Pᵢᵣ to Pᵢₗ in the calculation formula of Rᵣ_{_{—}}_{callus}. FIG. 8B shows a schematic view illustrating the risk adjustment factors of plantar pressures in FIG. 6, according to an exemplary embodiment.

The toe joint abnormality may be derived by the drift extreme value of the COP in the X-direction, so that the recorded drift distance of COP for the plantar pressure of each walking step in the Y-direction is defined as the front toe ROM risk adjustment factor. The toe structural abnormal risk adjustment factor is defined as the accumulated pressure value of measuring the toe location. Take FIG. 6 as an exemplar, the toe location is defined as the 12 locations of the plantar pressure sensors of the first three rows of the insole, and thus 12 pressure value p₁ to p₁₂ of the plantar pressure signals are defined accordingly. Therefore, the structural abnormal risk adjustment factor of the front toe may be calculated as follows:

$$\sum P = {\sum }_{i = 1}^{12} ( {p}_{i} \times Δ t ) ,$$

wherein Δt represents the time difference of before and after capturing the pressure signal, and the unit is second; and pᵢ represents the obtained value of plantar pressure signal Pi from plantar pressure sensor i.

The accumulated value of ΣP of plantar pressure signals may be used to assess whether the user has entered a high-risk area of tissue damage through the relationship diagram of pressure-withstanding versus time in FIG. 2. The risk adjustment factors COPₓ, COP_{y}, Rᵣ_{_{—}}_{callus}, Rₗ_{_{—}}_{callus}, Rₜₒₑ etc., may be used to represent the plantar soft tissue and circulatory status of the user and may be used to adjust the relationship diagram of pressure-withstanding versus time of the human body pressure ulcer as shown in FIG. 7.

The risk adjustment factors in the exemplary embodiments may be derived by the features values of the extremity pressure signals through a machine learning system. And the feature values of the extremity pressure signals, such as mentioned above, may be derived by one or more features of at least one extremity pressure center moving curve, at least one extremity pressure-time change, at least one limb local pressure performance, and at least two different extremity pressure performances.

According to an exemplary embodiment of the present disclosure, the pressure sensors may be connected to the electronic transceiver device or the mobile device, to create an interactive management system for damage risk of tissue pressure. The interactive management system for damage risk of tissue pressure may comprise pressure sensors, a wireless data transmission device, and an information processing device for assessing damage risk of tissue. The pressure sensors may be flexible electronic devices, and use wireless or wired communication to connect the electronic transceiver device or the mobile device.

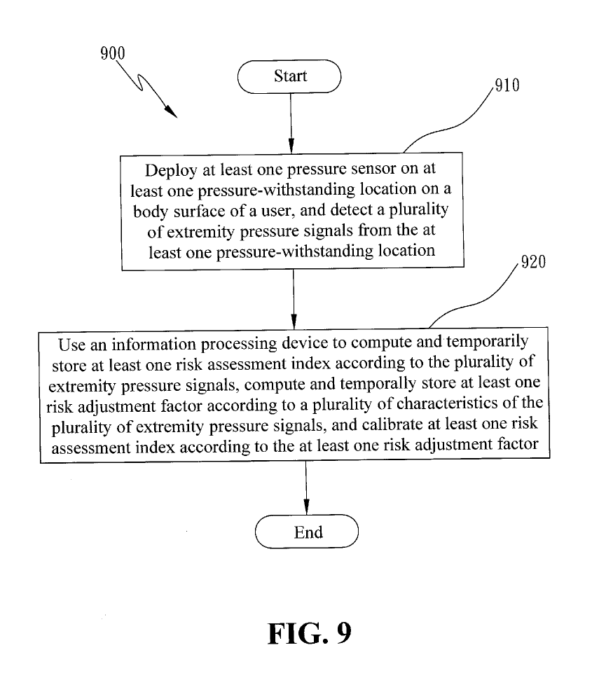

FIG. 9 shows a management method for damage risk of tissue pressure, according to an exemplary embodiment. Referring to FIG. 9, the risk management method for damage risk of tissue pressure 900 firstly deploys at least one pressure sensor on at least one pressure-withstanding location on a body surface of a user, and detects a plurality of extremity pressure signals from the at least one pressure-withstanding location (step 910). And, the method uses an information processing device to compute and temporarily store at least one risk assessment index according to the plurality of extremity pressure signals, compute and temporally store at least one risk adjustment factor according to a plurality of features of the plurality of extremity pressure signals, and calibrate at least one risk assessment index according to the at least one risk adjustment factor (step 920). The method may continuously collect more extremity pressure signals. When the time of collecting these extremity pressure signals reaches a predetermined recording time interval, the method outputs at least one risk assessment index and at least one risk adjustment factor.

The method 900 may use a pressure capture element to record the plurality of extremity pressure signal sensed by at least one pressure sensor. The pressure capture element begins recording at least one extremity pressure signal value when at least one extremity pressure signal appearing from the at least one pressure sensor until the there is one extremity pressure signal sensed by the at least one pressure sensor. The at least one pressure sensor may be manufactured by flexible electronic technologies and connected to an electronic receiving device or an mobile device, etc. with a wireless or a wired communication.

After the extremity pressure signals disappear, the method 900 may use an information processing device to compute at least one risk assessment index, such as the accumulated value of the extremities pressure signals or a maximum extremity pressure value, and the one or more risk adjustment factors, such as the COPₓ, the COP_{y}, the local pressure concentrated factor, the extremity structural abnormal risk factor, etc. The calculation of the risk assessment index and the one or more risk adjustment factor is not restated here. When the time of continuously collecting more extremity pressure signals reaches a predetermined recording time interval, the method outputs the at least one risk assessment index and the at least one risk adjustment factor. The information processing device may calculate a risk level of the user exposed to the pressure ulcer according to the cumulated value of the extremities pressure signals and the extremity pressure risk adjustment factor. The method 900 may output the aforementioned pressure risk level to a display device through a transmission device, such as a computer, a smart phone, a tablet PCs, or a smart electronic watch, and so on.

the exemplary embodiments of the present disclosure provide a management system and method for damage risk of tissue pressure, and its technology deploys at least one pressure sensor on pressure-withstanding location on a body surface of a user, and an information processing device connects to the at least one pressure sensor. The exemplary embodiments may use pressure peak value(s) or the accumulated value of pressures vs. time to calculate the tissue damage risk assessment index, and according to the features of the pressure signals or the physiological signals to calculate risk adjustment factor. And then may use one or more risk adjustment factors to adjust at least one tissue damage risk assessment index. The at least one tissue damage risk assessment index and the one or more risk adjustment factors may be transmitted out through such as a communication device. The information processing device may calculate a risk level of the user exposed to pressure ulcers according to the accumulated value of the extremity pressure signals and the at least one extremity pressure risk adjustment factor.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A management system for damage risk of tissue pressure, comprising:
at least one pressure sensor deployed on at least one pressure-withstanding location on a body surface of a user, and the at least one pressure sensor detects a plurality of extremity pressure signals from the at least one pressure-withstanding location; and
an information processing device that computes and temporarily stores at least one risk assessment index according to the plurality of extremity pressure signals, computes at least one risk adjustment factor according to a plurality of features of the plurality of extremity pressure signals, and then calibrates the at least one risk assessment index according to the at least one risk adjustment factor.

2. The system as claimed in claim 1, wherein said information processing device connects to the at least one pressure sensor.

3. The system as claimed in claim 1, wherein said at least one risk assessment index is at least one extremity maximum pressure value of said user, and said at least one extremity maximum pressure value is obtained from a plurality of pressure peak values of a plurality of extremity pressure areas of said user.

4. The system as claimed in claim 1, wherein said plurality of features of said plurality of extremity pressure signals are derived by a machine learning system.

5. The system as claimed in claim 1, wherein said at least one pressure sensor is at least one flexible electronic device.

6. The system as claimed in claim 1, wherein said plurality of features are derived by at least one of features including at least one extremity pressure center moving curve, at least one extremity pressure time change, at least one limb local pressure performance, and at least two extremities pressure performance difference.

7. The system as claimed in claim 1, wherein said user is an animal having hard and soft tissues, and said at least one pressure-withstand location is at least one extremity location of said animal.

8. The system as claimed in claim 1, wherein said system outputs said at least one risk assessment index and said at least one risk adjustment factor when the time that said system continues collecting said plurality of extremity pressure signals reaches a predetermined recording time interval.

9. The system as claimed in claim 1, wherein said at least one risk adjustment factor is at least one of factors including a calculating pressure center location of an extremity of the user, a local pressure concentrated factor, and an extremity structural abnormal risk factor.

10. The system as claimed in claim 9, wherein said local pressure concentrated factor describes said user's circulative state abnormality and soft tissue hyperplasia, and said extremity structural abnormal risk factor describes said user's extremity structure abnormality and extremity joint abnormality.

11. A management method for damage risk of tissue pressure, comprising:
deploying at least one pressure sensor on at least one pressure-withstanding location on a body surface of a user, and detecting a plurality of extremity pressure signals from the at least one pressure-withstanding location; and
using an information processing device to compute and storing at least one risk assessment index according to the plurality of extremity pressure signals, and compute at least one risk adjustment factor by using a plurality of features of the plurality of extremity pressure signals, and then calibrate the at least one risk assessment index according to the at least one risk adjustment factor.

12. The method as claimed in claim 11, wherein said method outputs said at least one risk assessment index and said at least one risk adjustment factor when the time that said method continues collecting said plurality of extremity pressure signals reaches a predetermined recording time interval.

13. The method as claimed in claim 11, wherein said method uses a pressure capture element to record said plurality of extremity pressure signals sensed by said at least one pressure sensor.

14. The method as claimed in claim 11, wherein said pressure capture element begins recording said at least one extremity pressure signal value once said at least one extremity pressure signal appears from said at least one pressure sensor until there is no extremity pressure signal sensed by said at least one pressure sensor.

15. The method as claimed in claim 11, wherein said information processing device calculates a risk level of the user exposed to a pressure ulcer according to said at least one risk assessment index and said at least one risk adjustment factor.

16. The method as claimed in claim 11, wherein said at least one risk assessment index is an accumulated value of extremity pressure signals or a maximum extremity pressure value of said user.

17. The method as claimed in claim 11, wherein said at least one risk adjustment factor is at least one of factors including a calculating pressure center location of an extremity of the user, a local pressure concentrated factor, and an extremity structural abnormal risk factor.

18. The method as claimed in claim 14, wherein when said at least one extremity pressure signal disappears, said method uses said information processing device to calculate said at least one risk assessment index and said at least one risk adjustment factor.
